(19) United States
(12) Patent Application Publication
(10) Pub. No.: US 2020/0327982 A1
(43) Pub. Date: Oct. 15, 2020

(54) INFORMATION MANAGEMENT SYSTEM AND RECEIVING APPARATUS

(71) Applicant: Canon Medical Systems Corporation, Otawara-shi (JP)

(72) Inventors: Akihiro TSUKAMOTO, Yokohama (JP); Kazushige HATORI, Saitama (JP); Hiromasa YAMAGISHI, Otawara (JP); Yosuke YANAGIDA, Nasushiobara (JP)

(73) Assignee: Canon Medical Systems Corporation, Otawara-shi (JP)

(21) Appl. No.: 16/842,843

(22) Filed: Apr. 8, 2020

(30) Foreign Application Priority Data
Apr. 11, 2019 (JP) 2019-075668

(51) Int. Cl. G16H 40/20; G16H 40/40; G16H 10/60

(52) U.S. Cl. CPC G16H 40/20; G16H 10/60; G16H 40/40

(57) ABSTRACT

According to one embodiment, an information management system includes an acquisition apparatus, a commonalization apparatus and a receiving apparatus. The acquisition apparatus acquires data stored in an external storage. The commonalization apparatus converts the acquired data into a preliminarily set commonalization format. The receiving apparatus receives the data converted into the commonalization format and sets, in the received data, a workflow status capable of branching along a transition of a person in charge of checking data in accordance with a status of the data.

### CROSS-REFERENCE TO RELATED APPLICATIONS

This application is based upon and claims the benefit of priority from the prior Japanese Patent Application No. 2019-075668, filed Apr. 11, 2019, the entire contents of which are incorporated herein by reference.

### FIELD

Embodiments described herein relate generally to an information management system and a receiving apparatus.

### BACKGROUND

When guidance necessary for medical treatment is made for a patient who uses an implantable cardiac pacemaker, the management fee for the guidance of the implantable cardiac pacemaker can be calculated once a month only. Furthermore, when the guidance necessary for medical treatment is made for the patient by means of remote monitoring for a period from the month following the last consultation month to the month before this consultation month, a predetermined number of points may be added to the management fee of the cardiac pacemaker as an addition for remote monitoring.

In remote monitoring, for example, first, hospital staff enters and sets a date, day of week, and time on which remote monitoring data is to be prepared on a server of the device maker, or given alert conditions from a dedicated Web site opened by a device maker of the cardiac pacemaker. A monitoring device associated with the cardiac pacemaker always communicates with the cardiac pacemaker and transmits data obtained through the cardiac pacemaker to the server of the device maker, usually once a day while patient is sleeping. Furthermore, the monitoring device also transmits the data obtained through the cardiac pacemaker to the server of the device maker when a given alert is detected through round-the-clock monitoring. Resulting PDF data of the remote monitoring is prepared on the server of the device maker at the timing of date, day of week, and time at which the above-mentioned set data is to be transmitted or at the timing of detecting the set alert conditions. Hospital staff accesses the dedicated Web site as necessary, refers to resulting data on the patient stored in the server, or downloads the resulting data in the PDF format to refer to it.

However, it is extremely time-consuming to access a dedicated Web site of a device maker to select an intended patient and refer to necessary resulting data, or to download PDF data as a remote monitoring result of the patient when necessary. This involves a problem wherein it is difficult to efficiently manage downloaded data.

### BRIEF DESCRIPTION OF THE DRAWINGS

FIG. 1 is a block diagram showing a functional configuration of an information management system according to a first embodiment.

FIG. 2 is a diagram showing an example of a structure of data mapped in the cloud system shown in FIG. 1.

FIG. 3 is a block diagram showing a functional configuration of a receiving apparatus shown in FIG. 1.

FIG. 4 is a diagram showing an example of a structure of XML data set by the data setting function shown in

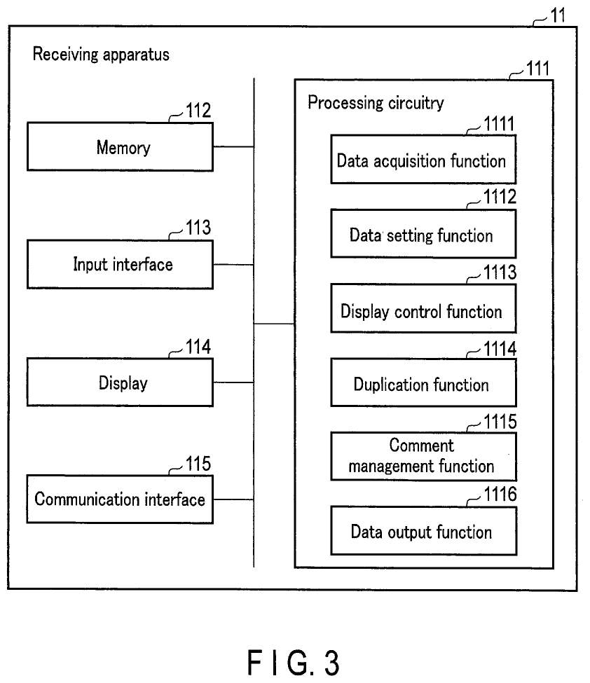

FIG. 3.

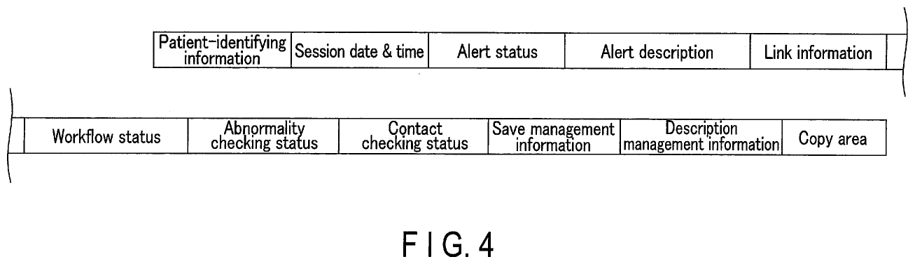

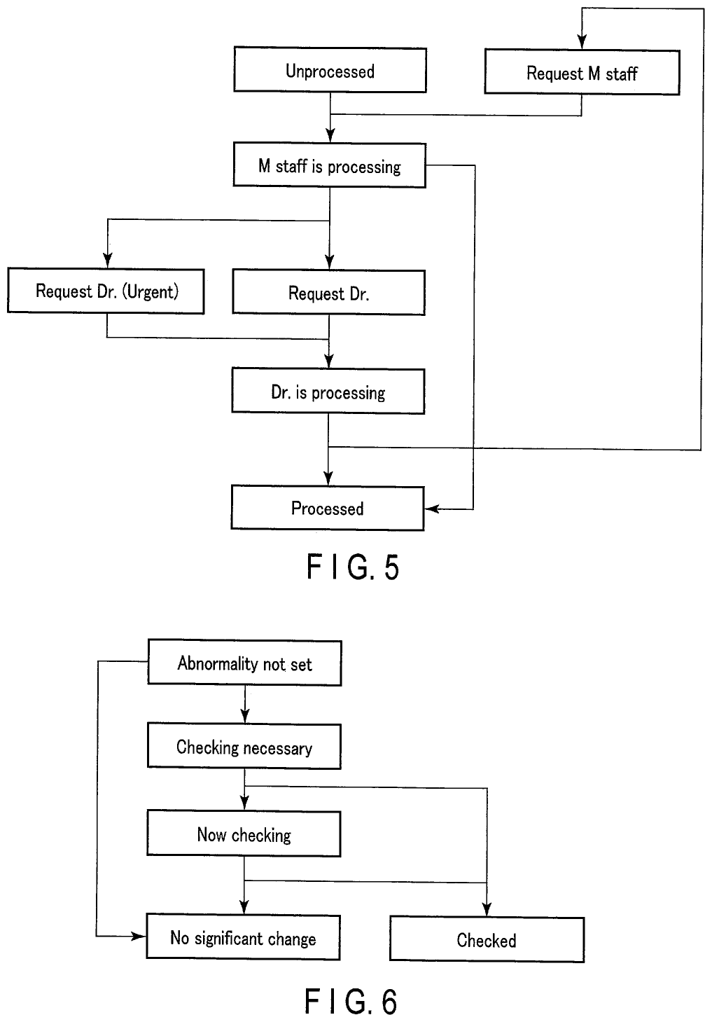

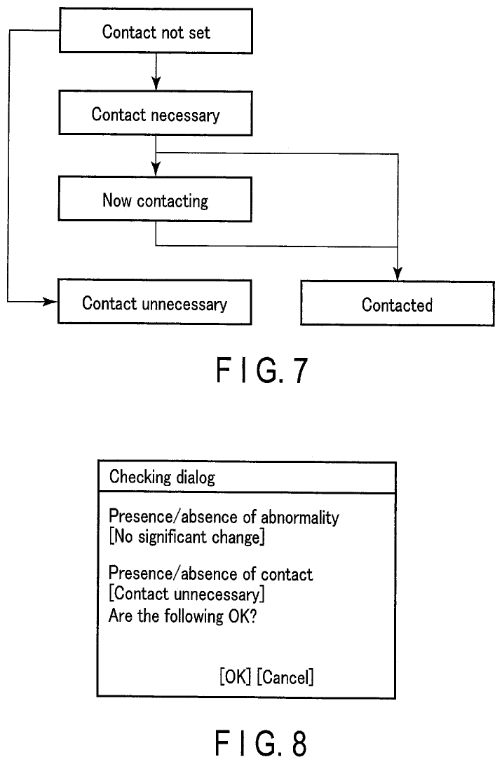

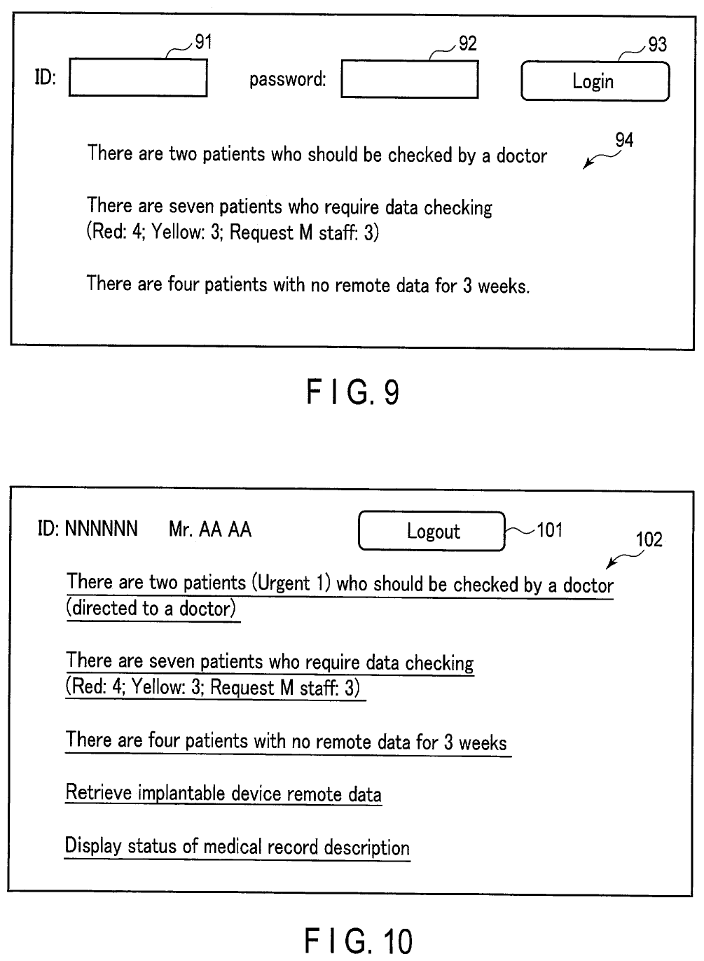

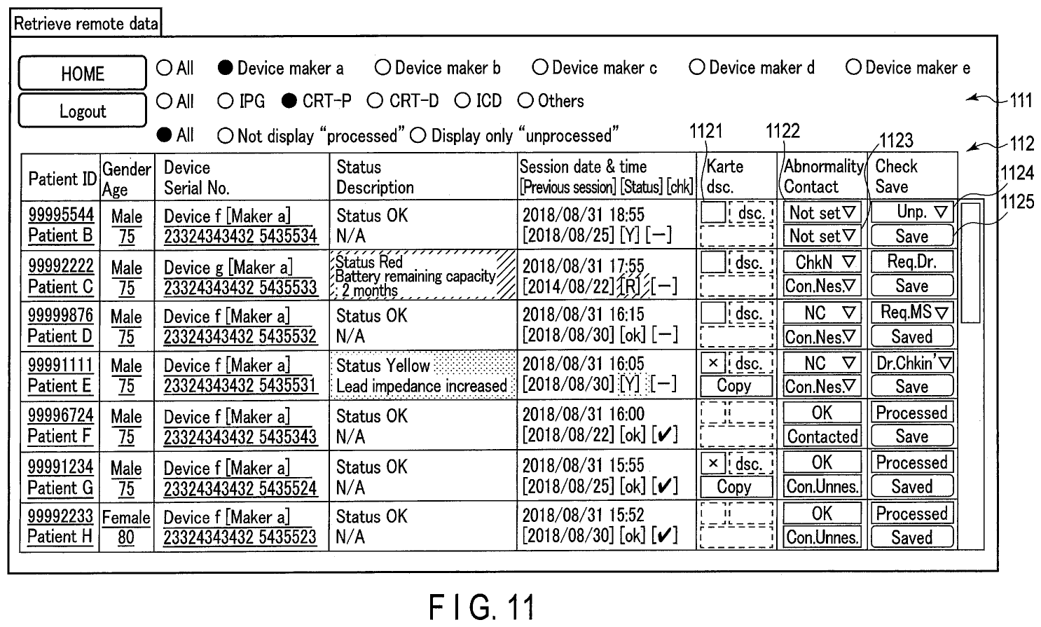

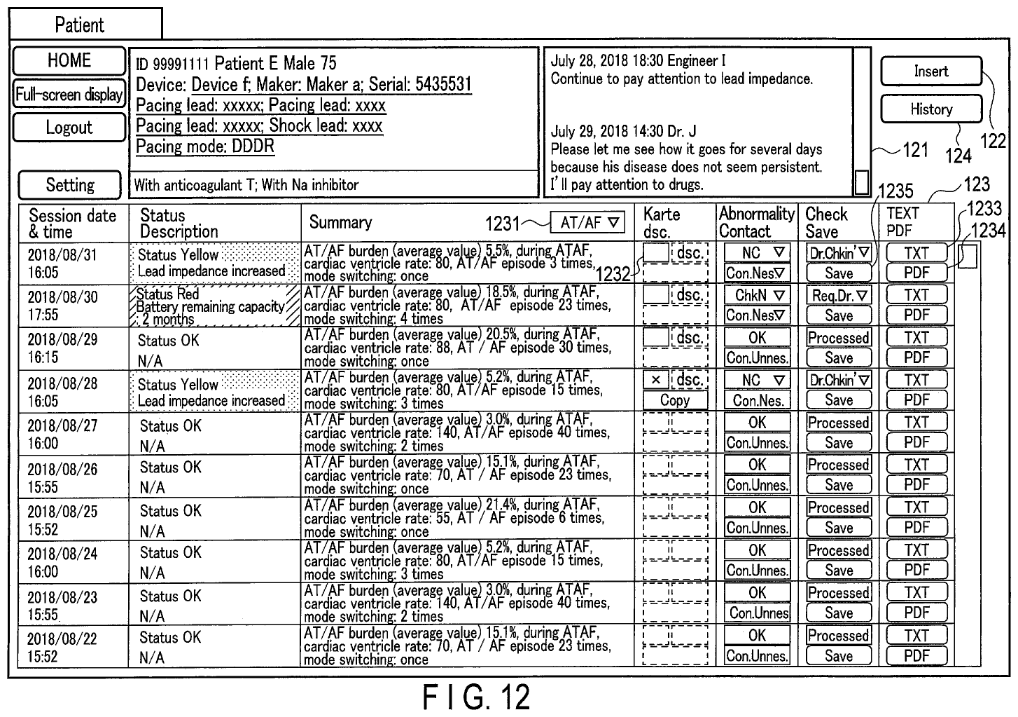

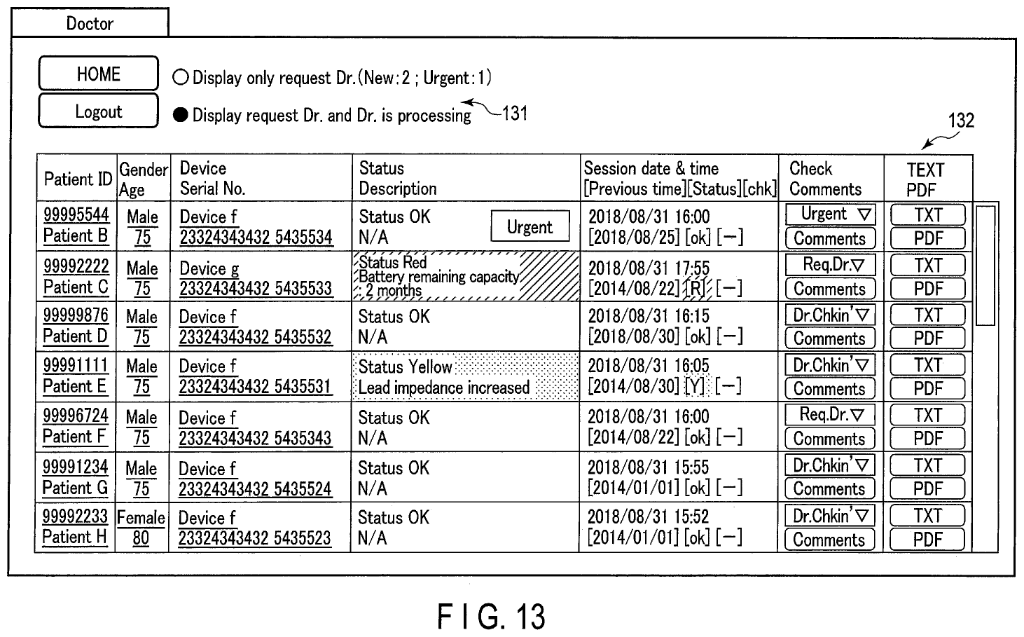

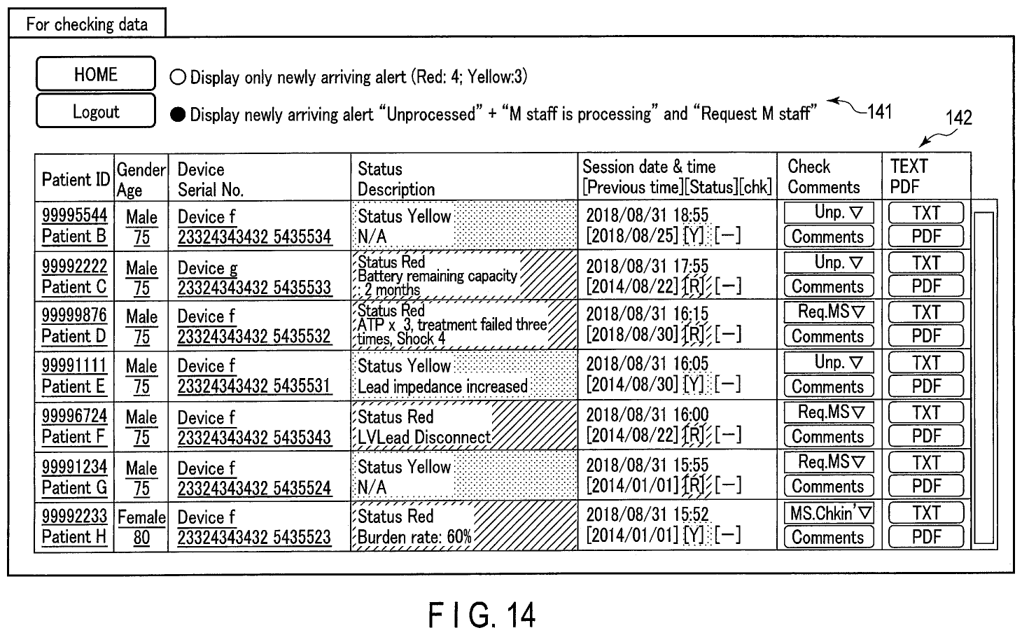

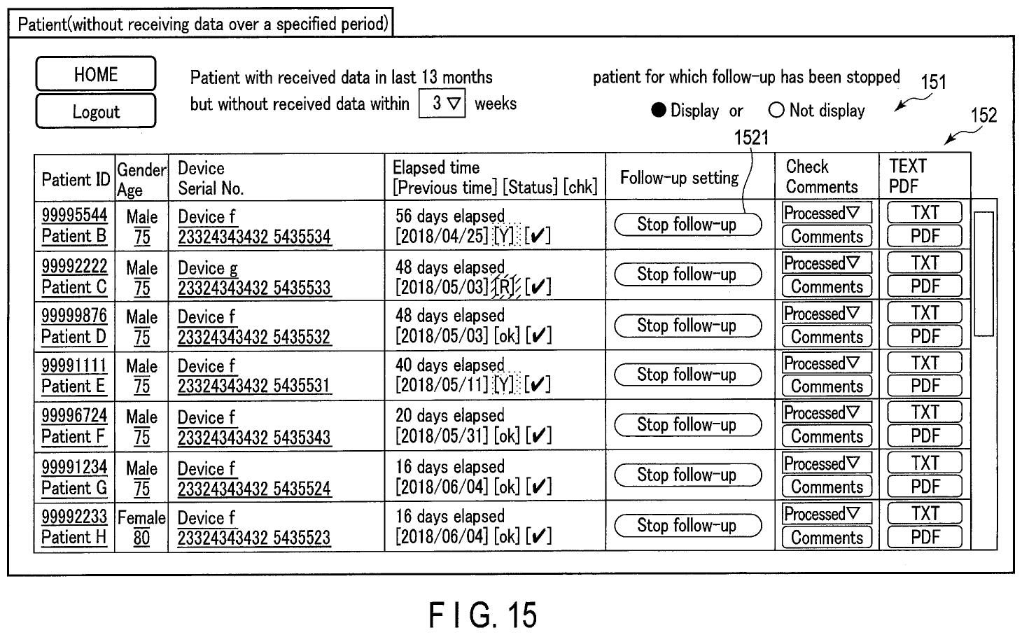

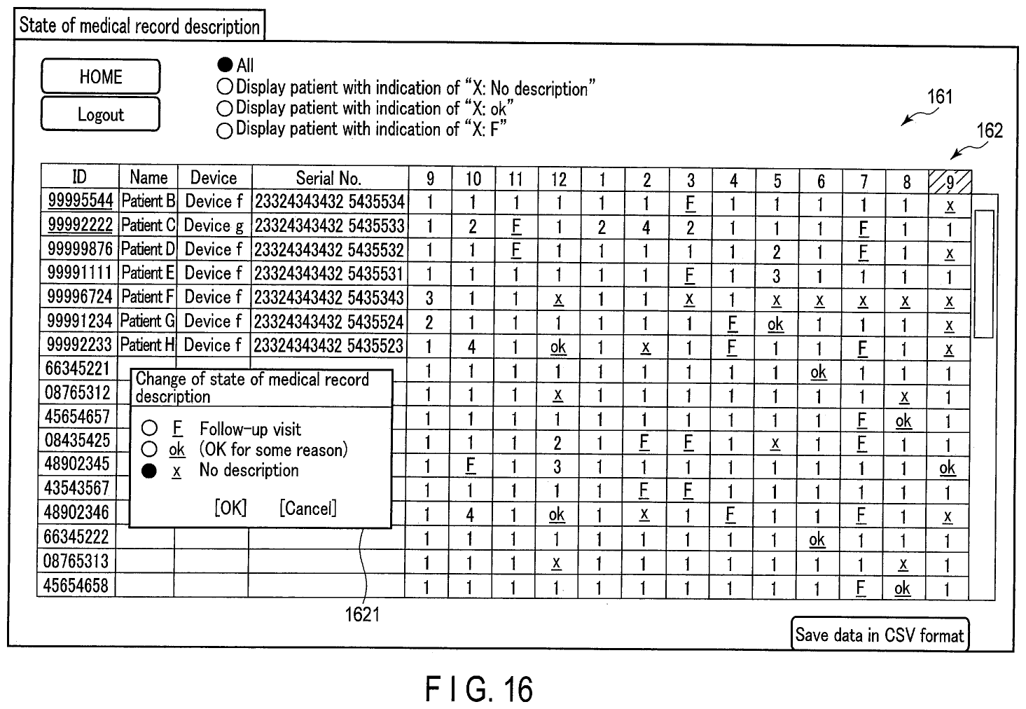

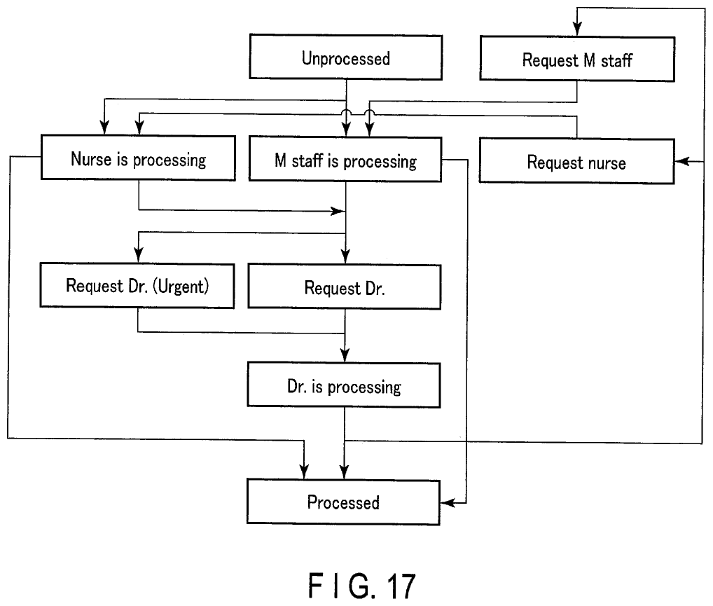

FIG. 5 is a diagram showing the setting of a workflow status shown in FIG. 4.

FIG. 6 is a diagram showing the setting of an abnormality checking status shown in FIG. 4.

FIG. 7 is a diagram showing the setting of a contact checking status shown in FIG. 4.

FIG. 8 is a diagram showing a checking dialog.

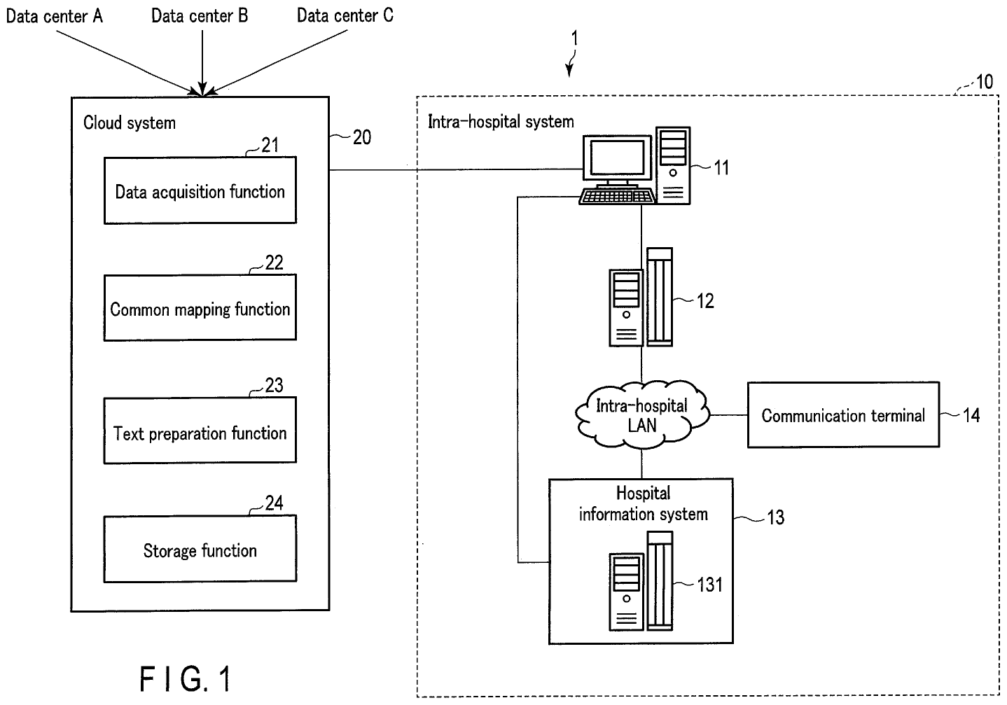

FIG. 9 is a diagram showing a login screen displayed on a display of the receiving apparatus shown in FIG. 1.

FIG. 10 is a diagram showing a home screen transitioned from a login screen shown in FIG. 9.

FIG. 11 is a diagram showing a data acquisition screen transitioned from the home screen shown in FIG. 10.

FIG. 12 is a diagram showing an individual screen for each patient, transitioned from the data acquisition screen shown in FIG. 11.

FIG. 13 is a diagram showing a screen for doctors, transitioned from the home screen shown in FIG. 10.

FIG. 14 is a diagram showing a data checking screen transitioned from the home screen shown in FIG. 10.

FIG. 15 is a diagram showing a no-data patient checking screen transitioned from the home screen shown in FIG. 10.

FIG. 16 is a diagram showing a description status checking screen transitioned from the home screen shown in FIG. 10.

FIG. 17 is a diagram showing the setting of a workflow status in the case where a nurse is treated as staff independent of medical staff.

### DETAILED DESCRIPTION

In general, according to one embodiment, an information management system includes an acquisition apparatus, a commonalization apparatus and a receiving apparatus. The acquisition apparatus acquires data stored in an external storage. The commonalization apparatus converts the acquired data into a preliminarily set commonalization format. The receiving apparatus receives the data converted into the commonalization format and sets, in the received data, a workflow status capable of branching along a transition of a person in charge of checking data in accordance with a status of the data.

Hereinafter, the information management system and the receiving apparatus according to the present embodiment will be described with reference to the drawings. In the following embodiments, duplication of descriptions on portions provided with the same reference numbers will suitably be omitted, assuming that those portions perform the same operation. Hereinafter, a first embodiment will be described using drawings.

### First Embodiment

FIG. 1 is a block diagram showing an example of a functional configuration of an information management system 1 according to a first embodiment. The information management system 1 shown in FIG. 1 includes an intra-hospital system 10 and a cloud system 20. The intra-hospital system 10 and the cloud system 20 are connected via a dedicated line, for example.

The information management system 1 is a network system that complies with the guidelines at the time of storing medical information in a public cloud (system), etc., for example, three guidelines published by the three Ministries of the Ministry of Health, Labor and Welfare, the Ministry of Economy, Trade and Industry, and the Ministry of Internal Affairs and Communications, the so-called 3 Guidelines published by three Ministries. That is, the information management system 1 is a network system that complies with “Security Guidelines for Medical Information Systems” established by the Ministry of Health, Labor and Welfare, “Security Guidelines for Information Processing Business Proprietors who are entrusted with medical information and manage the information” established by the Ministry of Economy, Trade and Industry, and “Security Guidelines when cloud service business proprietors deal with medical treatment information” established by the Ministry of Internal Affairs and Communications.

The cloud system 20 acquires data stored in a data center installed for each device maker and converts the acquired data into a preliminarily set shared common commonalization format to store the data. The data center is an example of external storage and may be referred to as “a data server”. The cloud system 20 is a cloud computing system in which a given function is shared among a plurality of devices via a network and realized in collaboration, for example. A realized example of the acquisition function is not limited to the cloud system 20 and may be realized by a server which is placed in, for example, an intra-hospital system and connected to a data center of the device maker via a line where the security is ensured, or a server which communicates with an intra-hospital system placed in an area where the security is ensured, while a DeMilitarized Zone (DMZ), etc. being set outside the intra-hospital system, or a data center, by means where security is secured.

Data which is acquired by cardiac pacemakers manufactured by each device maker and implanted in patients intended for remote monitoring is stored in a data center installed for the device maker. For example, a monitoring device associated with a cardiac pacemaker always communicates with the cardiac pacemaker and transmits the data acquired by the cardiac pacemaker to a data center of the device maker, usually once a day, for example, while hospital staff is sleeping. The monitoring device also transmits the data acquired by the cardiac pacemaker to the data center of the device maker when detecting a given alert through round-the-clock monitoring.

The data center prepares resulting data of the remote monitoring based on the data received from the monitoring device at a given frequency or prepares resulting data of remote monitoring when the received data satisfies a given requirement.

In the cloud system 20, a plurality of devices take partial charge of roles and work together via a network, and thereby the cloud system 20 has a data acquisition function 21, a common mapping function 22, a text preparation function 23, and a storage function 24.

The data acquisition function 21 is a function to acquire data stored in data centers A to C provided for each device maker from the data centers A to C and is an example of the acquisition apparatus. Specifically, communication software of each device maker is installed in, for example, a tenant for a hospital according to the present embodiment within the cloud system 20. From among a plurality of processors included in the cloud system 20, a processor corresponding to the tenant executes the communication software, thereby accessing the data centers A to C at a given cycle and acquiring data stored in the data centers A to C. The data center accessed by the data acquisition function 21 may be a single data center operated by a single device maker or a plurality of data centers respectively operated by a plurality of device makers.

The data transmitted from the data centers A to C to the cloud system 20 complies with a preliminarily set standard, for example. The preliminarily set standard is a Health Level 7 (HL7) described using, for example, Implantable Device Cardiac Observation (IDCO) set up by Integrating the Healthcare Enterprise (IHE). The preliminarily set standard is not limited to HL7, provided that it is a standard for exchange of information. The data transmitted in compliance with HL7 includes various pieces of information relating to remote monitoring, for example, patient information relating to patients to be monitored, measurement information relating to measurements, and the like.

The patient information includes, for example, patient-identifying information for identifying a patient. The patient-identifying information may be a name of a patient or a given number. The information included in the patient information is not limited thereto. The patient information may include, for example, a maker name indicating a name of a device maker, a device name indicating a name of a device, a type of a device, a serial number of a device, and the like.

The measurement information includes, for example, a session date and time indicating a session start time of a data recording period (referred to as a “session”) included in data transmitted from a cardiac pacemaker to a monitoring device, the session duration time, an alert status indicating an alert level based on the data transmitted from the cardiac pacemaker, an alert description for describing an alert transmitted from the cardiac pacemaker, measurement items measured by the cardiac pacemaker, measured values of the measurement items, and the like. The information included in the measurement information is not limited thereto. For example, the information does not necessarily include all the above-mentioned information, or new information may be added to the information. The session date and time, session duration time, alert status, alert description, measurement items, measured items, measured values, etc. included in one-time transmission are not limited to data acquired from the cardiac pacemaker within one-time session. Data of a plurality of sessions may be included in one-time transmission.

Examples of the measurement items measured by the cardiac pacemaker include parameters, such as AF/AT, a pacing rate, and lead-sensing information. The measurement items may include a statistic result based at least on any of measurement results.

Detailed information describing measured information on a patient to be monitored in detail may be attached to data transmitted from the data centers A to C. The detailed information may include information measured in the past (generally, information and/or a graph relating to a trend indicating long-term transitions). The detailed information is attached in the form of a data file in a PDF format.

The common mapping function 22 is a function to map data transmitted from a data center in a common format and is an example of a commonalization apparatus. For example, data transmitted from each data center A to C complies with HL7; however, the way to comply with HL7 may sometimes differ on a device maker basis in a strict sense, depending on the different functions or set items among respective devices. From among the processors included in the cloud system 20, a processor corresponding to a facility-based tenant converts data acquired from the data centers A to C into data which complies with IDCO and that is in the format preliminarily set in the cloud system 20 (in the present embodiment, for example, an XML format described based on IDCO is used).

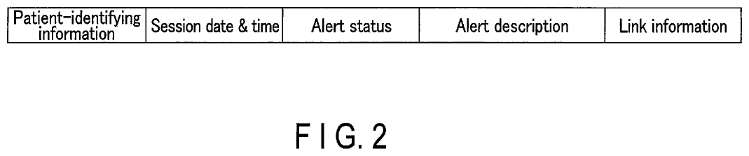

FIG. 2 is a pattern diagram showing a structural example of data mapped by the cloud system 20 shown in FIG. 1. The data shown in FIG. 2 includes, for example, the items patient-identifying information, session date & time, alert status, alert description, and PDF data and/or linked information of text data to be described later. The items included in mapped data are not limited thereto. The mapped data may include items of a maker name, a device name, a device type, a serial number, and the like. The sequential order of the items included in the mapped data is not limited thereto.

Furthermore, the processor corresponding to the facility-based tenant associates a measured value for each measurement item of data transmitted from the data centers A to C with, for example, patient-identifying information and session date & time, and writes in a data table stored in the cloud system 20. The processor may also convert the measured value for each measurement item transmitted from the data centers A to C into, for example, XML format data which complies with IDCO.

The text preparation function 23 is a function to prepare text data based on the data after being subjected to common mapping processing. Specifically, for example, from among a plurality of processors included in the cloud system 20, the processor corresponding to the facility-based tenant prepares text data based on the data mapped in a common format and the data written in a data table.

The storage function 24 is a function to store data after being subjected to the common mapping processing. Specifically, from among the plurality of processors included in the cloud system 20, the processor corresponding to the facility-based tenant stores the data mapped in the common format in a database constructed in the cloud system 20. In addition, the processor stores, in the database, the data table in which measured values are written. In addition, the processor stores, for example, detailed information transmitted in a PDF format in a database. Furthermore, the processor of the cloud system 20 stores text data prepared by the text preparation function 23 in the database.

The intra-hospital system 10 shown in FIG. 1 is a system constructed in a hospital. The intra-hospital system 10 includes, for example, a receiving apparatus 11, a department server 12, a hospital information system 13, and a communication terminal 14. The receiving apparatus 11 is connected to the cloud system 20 via a dedicated line and connected to the department server 12 and an HIS server 131 of the hospital information system (HIS) 13, respectively. The department server 12 and the HIS server 131 are data-communicatively connected via an intra-hospital network such as a local area network (LAN). The connection to the intra-hospital network may be a wired or wireless connection.

The department server 12 is a server used in a circulatory organ department information system. The circulatory organ department information system is a system that manages information relating to, for example, implanted devices in a circulatory organ department, which is one medical treatment department. The department server 12 stores data output from the receiving apparatus 11 in a memory provided in the inside of the department server 12.

FIG. 1 illustrates an example in the case where a single department server 12 is present; however, the number of the department servers 12 is not limited thereto. A plurality of the department servers 12 may be provided when necessary.

The hospital information system 13 is a system which manages information relating to electronic medical records, for example. The hospital information system 13 includes, for example, an HIS server 131. The HIS server 131 stores, in the hospital information system 13, information relating to electronic medical records. The HIS server 131 updates stored information based on information entered by, for example, a doctor.

FIG. 1 illustrates an example in the case where servers included in the hospital information system 13 are only the HIS server 131; however, the number of servers is not limited thereto. A plurality of the HIS servers 131 may be provided when necessary. For example, the HIS server 131 may be provided for each pieces of information managed. Specifically, the hospital information system 13 may include an electronic medical record system and an order-entry system, and a server may be provided for each system.

The communication terminal 14 is a terminal, for example, for a doctor to access a system, a server, and the like connected to a LAN in the hospital. FIG. 1 illustrates a case where the communication terminal 14 belongs to neither the hospital information system 13 nor the department information system; however, the configuration of the present embodiment is not limited thereto. The communication terminal 14 may belong to either one of the hospital information system 13 and the department information system, provided that a user, such as a doctor, can use it.

The receiving apparatus 11 acquires data stored in the cloud system 20 and displays, for doctors or medical staff, the acquired data in an efficient format. In the present embodiment, the medical staff includes, for example, clinical engineers who are specialized in medical devices, and nurses. FIG. 1 illustrates a case where only one receiving apparatus 11 is included in the intra-hospital system 10; however, the number of the receiving apparatus 11 is not limited thereto. A plurality of the receiving apparatuses 11 may be provided.

FIG. 3 is a block diagram showing an example of the functional configuration of the receiving apparatus 11 shown in FIG. 1. The receiving apparatus 11 shown in FIG. 3 includes a processing circuitry 111, a memory 112, an input interface 113, a display 114, and a communication interface 115. The processing circuitry 111, memory 112, input interface 113, display 114, and communication interface 115 are communicatively connected to each other via, for example, a bus.

The processing circuitry 111 is a processor that functions as a core of the receiving apparatus 11. The processing circuitry 111 realizes a function corresponding to a program stored in the memory 112, etc., by executing the program.

The memory 112 is a storage device which stores various kinds of information, such as a read only memory (ROM), a random access memory (RAM), a hard disk drive (HDD), a solid state drive (SSD), and an integrated circuit storage device, for example. The memory 112 may be a driving unit, etc. which reads and writes various kinds of information among portable storage media, such as a CD-ROM drive, a DVD drive, and a flash memory. The memory 112 is not necessarily realized by a single storage device. For example, the memory 112 may be realized by a plurality of storage devices. Furthermore, the memory 112 may be placed in another computer connected to the receiving apparatus 11.

The memory 112 stores a program, etc. according to the present embodiment. The program may be preliminarily stored in the memory 112, for example. In addition, a program may be stored and distributed in a non-transient storage medium and read from the non-transient storage medium and installed in the memory 112. Furthermore, data read out from the cloud system 20 and processed in the processing circuitry 111 is stored in the memory 112.

The memory 112 stores patient information on patients intended for remote monitoring. For example, the memory 112 stores, as patient information, a patient ID, a patient name, gender, a birth date, other information on the patient, a maker name, a device name, a device type, a serial number, comments, operation histories, a date on which the final data was acquired, a presence or absence of follow-up, and contents of measurement/statistic data displayed the previous time. The patient information is not limited thereto. For example, the patient information does not necessarily include all the above-mentioned information, and new information may be added to the patient information. The patient information is updated through instructions from the department server 12 or hospital information system 13, for example.

The input interface 113 accepts various kinds of input operations from a user and converts an accepted input operation into an electric signal and outputs the electric signal to the processing circuitry 111. The input interface 113 is connected to input devices, for example, a mouse, a keyboard, a trackball, a switch, a button, a joystick, a touch pad, or a touch panel in which instructions are entered by touching its operation screen surface. The input device connected to the input interface 113 may be an input device provided in another computer connected via a network, etc.

The display 114 displays various kinds of information in accordance with the instructions from the processing circuitry 111. With respect to the display 114, any display, for example, a Cathode Ray Tube (CRT) display, a liquid crystal display, an organic EL display, an LED display, and a plasma display, etc. may be suitably used.

The communication interface 115 performs data communications with the cloud system 20, the department server 12, and the HIS server 131. For example, the communication interface 115 performs data communications with the cloud system 20 in accordance with, for example, a discretionally selected standard utilizing a dedicated line. Furthermore, the communication interface 115 performs data communications with the department server 12 and the HIS server 131 in accordance with an already known and preliminarily set standard.

The processing circuitry 111 shown in FIG. 3 realizes a function corresponding to, for example, a program stored in the memory 112 by executing the program. For example, the processing circuitry 111 includes a data acquisition function 1111, a data setting function 1112, a display control function 1113, a duplication function 1114, a comment management function 1115, and a data output function 1116. In the present embodiment, a case will be described where the data acquisition function 1111, data setting function 1112, display control function 1113, duplication function 1114, comment management function 1115, and data output function 1116 are realized by a single processor; however, the configuration of the present embodiment is not limited thereto. For example, a plurality of independent processors may be combined together to configure processing circuitry such that each of the processors executes a program, thereby realizing the data acquisition function 1111, data setting function 1112, display control function 1113, duplication function 1114, comment management function 1115, and data output function 1116.

The data acquisition function 1111 is a function to acquire data stored in the cloud system 20 and is an example of the acquisition apparatus. Specifically, for example, in the data acquisition function 1111, the processing circuitry 111 acquires data in an XML format which complies with IDCO (hereinafter, referred to as “XML data”) stored in the cloud system 20 at a preliminarily set cycle, and data written in a data table. Furthermore, the processing circuitry 111 acquires PDF data or text data or both stored in the cloud system 20 in accordance with instructions entered by a doctor or medical staff member, etc., for example.

The data setting function 1112 is a function to set, in the acquired data, information on a given item and is an example of a setting unit. Specifically, for example, in the data setting function 1112, the processing circuitry 111 sets a checking status in the XML data acquired from the cloud system 20. The checking status is information for efficiently checking a current status of data, for example. The checking statuses include a workflow status for checking a workflow. The checking statuses may include an abnormality checking status for checking for the presence or absence of abnormality. Also, the checking statuses may include a contact checking status for checking for the presence or absence of a contact to a patient.

Furthermore, for example, the processing circuitry 111 may set management information in the XML data acquired from the cloud system 20. The management information is information for managing the presence or absence of implementation of a given operation for designated data or information for managing whether or not it is necessary to perform a given operation. The management information includes save management information on whether or not the designated data is saved in the department server 12 or hospital information system 13, and description management information for managing whether or not a medical record is described by a doctor.

For example, in the data setting function 1112, the processing circuitry 111 changes a checking status of the XML data or management information saved in the memory 112 in accordance with instructions entered by a doctor or medical staff member.

FIG. 4 is a pattern diagram depicting a structural example of XML data set by the data setting function 1112 shown in FIG. 3. The XML data shown in FIG. 4 has a structure in which checking statuses and management information are added to the data shown in FIG. 2. The XML data shown in FIG. 4 includes, as checking statuses, a workflow status, an abnormality checking status, and a contact checking status and includes, as management information, data-save management information, and description management information. The XML data includes, as the management information, save management information and description management information. Also, the XML data shown in FIG. 4 includes a copy area for inputting information that requires duplication. The items set by the data setting function 1112 are not limited to the items shown in FIG. 4. For example, not all of the workflow status, abnormal checking status, contact checking status, save management information, description management information, and copy area are necessarily included in the items.

FIG. 5 is a pattern diagram showing a setting example of the workflow status shown in FIG. 4. The setting example of the workflow status shown in FIG. 5 shows transitions of a person in charge of checking data within a hospital, i.e., a workflow. The direction of change in workflow status is not one direction in line with the transitions of the person in charge of checking data, and the direction may change in a branched manner in accordance with the status of data. For example, when acquiring data from the cloud system 20, the processing circuitry 111 executes the data setting function 1112 and sets the workflow status to “Unprocessed” as the default. The workflow status of “Unprocessed” transitions to “M staff is processing” in accordance with instructions entered by a medical staff member, for example. The workflow status of “M staff is processing” transitions to “Processed,” “Request Dr. (Urgent)” or “Request Dr.” in accordance with instructions entered by medical staff, for example. The workflow statuses of “Request Dr. (Urgent)” and “Request Dr.” transitions to “Dr. is processing” in accordance with instructions entered by the doctor, for example. The workflow status of “Dr. is processing” transitions to “Processed” or “Request M staff” in accordance with instructions entered by the doctor, for example. The workflow status of “Request M staff” transitions to “M staff is processing” in accordance with instructions entered by medical staff, for example. That is, the direction of the course of the transition route indicated by a workflow status is not one direction and may take a branched route in accordance with the status of data.

FIG. 6 is a pattern diagram showing a setting example of the abnormality checking status shown in FIG. 4. The setting example of the abnormality checking status shown in FIG. 6 indicates a route for checking an abnormality determined based on a parameter included in the data. For example, when acquiring data from the cloud system 20, the processing circuitry 111 executes the data setting function 1112 and sets the abnormality checking status to “Abnormality not set” as the default. The abnormality checking status of “Abnormality not set” transitions to “Checking necessary” or “No significant change” in accordance with instructions entered by medical staff, for example. The abnormality checking status of “Checking necessary” transitions to “Now checking” or “Checked” in accordance with instructions entered by a doctor, for example. The abnormality checking status of “Now checking” transitions to “No significant change” or “Checked” in accordance with instructions entered by the doctor, for example.

FIG. 7 is a pattern diagram showing a setting example of the contact checking status shown in FIG. 4. The setting example of the contact checking status shown in FIG. 7 indicates a route for checking whether or not contacting a patient is required determined based on a parameter included in the data. When acquiring data from, for example, the cloud system 20, the processing circuitry 111 executes the data setting function 1112 and sets the contact checking status to “Contact not set” as the default. The contact checking status of “Contact not set” transitions to “Contact necessary” or “Contact unnecessary” in accordance with instructions entered by a medical staff member, for example. The contact checking status of “Contact necessary” transitions to “Now contacting” or “Contacted” in accordance with instructions entered by a doctor, for example. The contact checking status of “Now contacting” transitions to “Confirmed” in accordance with instructions entered by the doctor, for example.

A doctor or medical staff member may not independently enter instructions for each of the workflow status, the abnormality checking status, and the contact checking status. For example, entries in the workflow status, abnormality checking status, and contact checking status are linked together, and these statuses may be set such that some of the entries can be shortcut.

For example, when the processing circuitry 111 changes the workflow status to “Processed” in accordance with instructions of a doctor or medical staff member, the processing circuitry 111 checks the abnormality checking status and the contact checking status. When the abnormality checking status is neither “No significant change” nor “Checked” and the contact checking status is neither “Contact unnecessary” nor “Contacted”, the processing circuitry 111 may use the abnormality checking status and the contact checking status as given settings, for example. For example, the processing circuitry 111 may set the abnormality checking status to “No significant change” and may set the contact checking status to “Contact unnecessary”. At that time, the processing circuitry 111 causes the display 114 to display a checking dialog shown in FIG. 8, and when “OK” is entered, the processing circuitry 111 may actually set the abnormality checking status to “No significant change” and set the contact checking status to “Contact unnecessary”.

For the save management information shown in FIG. 4, “Unsaved” is set as the default. When data is saved by a doctor or medical staff member, a statement to that effect is entered as “saved” in the save management information.

In the description management information, “Not conducted” is set as the default. When a description in a medical record is conducted by a doctor or medical staff member, a statement to that effect is entered as “Conducted” in the description management information. When “Conducted” is entered in the description management information, the duplication function 1114 is executable, for example.

The display control function 1113 is a function to display desired information on the display 114 based on the data stored in the memory 112 and is an example of a display controller. Specifically, for example, in the display control function 1113, the processing circuitry 111 displays data with contents desired by a doctor or medical staff member, in accordance with instructions entered from the doctor or medical staff member. In addition, the processing circuitry 111 classifies data stored in the memory 112 and extracts desired data from the data stored in the memory 112, in accordance with instructions entered by a doctor or medical staff member.

The duplication function 1114 is a function to prepare information which requires duplication. Specifically, for example, in the duplication function 1114, the processing circuitry 111 prepares text information which requires duplication and inputs the prepared text information in a copy area, in accordance with instructions entered by a doctor or medical staff member.

The comment management function 1115 is a function to manage comment information. Specifically, for example, in the comment management function 1115, the processing circuitry 111 prepares comment information and inputs the prepared comment information in patient information stored in the memory 112, in accordance with instructions entered by a doctor or medical staff member.

The data output function 1116 is a function to output data acquired from the cloud system 20 to a given output destination. Specifically, for example, in the data output function 1116, the processing circuitry 111 outputs text data acquired from the cloud system 20 to a given destination, e.g., the memory 1112, in a file format or an already known format that has been preliminarily set, for example, a comma separated value (CSV) format, in accordance with instructions entered by a doctor or medical staff member. Also, the processing circuitry 111 outputs PDF data acquired from the cloud system 20 to a given destination, in a file format in accordance with instructions entered by a doctor or medical staff member. When outputting the text data, PDF data, or both of them, the processing circuitry 111 inputs “Saved”, indicating that the data is saved, in the save management information through the data setting function 1112, for example.

Next, the operations of the receiving apparatus 11 provided in the information management system 1 configured as described above will be described in detail. FIGS. 9 to 16 are pattern diagrams each showing an example of a display screen displayed on the display 114 of the receiving apparatus 11 in accordance with an operation of the receiving apparatus 11.

FIG. 9 is a pattern diagram showing an example of a display screen of the display 114 when a program according to the present embodiment is started in the receiving apparatus 11 shown in FIG. 1. The display screen shown in FIG. 9 is a login screen. An entry box 91 for entering an ID of a doctor or medical staff member, an entry box 92 for entering a password of the doctor or medical staff member, and a login button 93 are displayed on the login screen.

In addition, items to be addressed on a priority basis are displayed in a display area 94 of the login screen. On the display screen shown in FIG. 9, for example, “There are two patients who should be checked by a doctor,” “There are five patients who require data checking (Red: 2; Yellow: 3; Request M staff: 3),” and “There are four patients with no remote data for 3 weeks” are displayed as items to be addressed on a priority basis.

The display of the items to be addressed on a priority basis is realized by the display control function 1113 of the processing circuitry 111, for example. For example, for the display of “There are two patients who should be checked by a doctor,” the processing circuitry 111 records the number of pieces of session data with a workflow status set to “Request Dr. (Urgent)” or “Request Dr.” out of the session data stored in the memory 112. The processing circuitry 111 prepares text information for displaying “There are two patients who should be checked by doctor” based on the recorded numerical value.

For example, for the display of “There are five patients who require data checking (Red: 2, Yellow: 3, Request M staff: 3),” the processing circuitry 111 records the number of pieces of data with a workflow status set to “Request M staff” and the number of pieces of data with an alert status indicating an alert level determined to be “Red” or “Yellow” from among the session data stored in the memory 112. The processing circuitry 111 prepares text information for displaying “There are five patients who require data checking (Red: 2, Yellow: 3, Request M staff: 3)” based on the recorded numerical values.

Furthermore, for example, for the display of “There are four patients with no remote data for 3 weeks,” the processing circuitry 111 reads out, for example, the last data acquisition date of the patient information stored in the memory 112. The processing circuitry 111 calculates a period with no remote data on a patient-by-patient basis from the read out last data acquisition date and the current date. The processing circuitry 111 records the number of pieces of session data in which the calculated period exceeds three weeks. The processing circuitry 111 prepares text information for displaying “There are four patients with no remote data for 3 weeks” based on the recorded numerical value. The numerical value of the period in which no remote data is present may be set in a discretional manner.

According to the login screen shown in FIG. 9, the items to be checked can be checked at a glance even before a user logs in to the receiving apparatus. The doctor or medical staff member who uses the receiving apparatus 11 enters his or her ID and password in the entry boxes 91 and 92 respectively on the login screen and presses a login button 93. With this, the display screen transitions to the home screen.

FIG. 10 is a pattern diagram showing an example of a home screen transitioned from the login screen shown in FIG. 9. An ID of a doctor or medical staff member: NNNNNN, the name of the doctor or medical staff member: Mr. AA AA, and a logout button 101 are displayed on the home screen.

The items to be addressed on a priority basis and items relating to routine tasks are displayed in a display area 102 of the home screen. Specifically, in the display area 102, for example, “There are two patients who should be checked by a doctor (Urgent 1) (intended for doctor),” “There are seven patients who require data checking (Red: 4; Yellow: 3; Request M staff: 3),” and “There are four patients with no remote data for 3 weeks” are displayed as items to be addressed on a priority basis. The display of these records is realized through, for example, the display control function 1113 of the processing circuitry 111, similarly to the login screen shown in FIG. 9. In addition, in the display area 102, for example, “Retrieve implantable device remote data” and “Display status of medical record description” are displayed as items relating to routine tasks.

A link for transitioning to a display corresponding to each item is attached to the display items displayed in the display area 102. That is, a link for transitioning to a screen for a doctor is attached to “There are two patients who should be checked by a doctor (Urgent 1) (intended for doctor).” A link for transitioning to a data checking screen is attached to “There are seven patients who require data checking (Red: 4; Yellow: 3; Request M staff: 3).” A link for transitioning to a screen for checking patients with no data is attached to “There are four patients with no remote data for 3 weeks.” A link for transitioning to a data retrieval screen is attached to “Retrieve implantable device remote data”. Furthermore, a link for transitioning to the description status checking screen is attached to “Display status of medical record description”.

A doctor or medical staff member who uses the receiving apparatus 11 selects, for example, an item corresponding to a desired task on the home screen shown in FIG. 10. With this, the screen is transitioned from the home screen to each screen for conducting a desired task.

FIG. 11 is a pattern diagram showing an example of a data retrieval screen transitioned from the home screen shown in FIG. 10. At least part of the session data stored in the memory 112 is displayed on the data retrieval screen.

The selection items for selecting an extraction target are displayed in a first display area 111 on the data retrieval screen. Specifically, in the first display area 111 shown in FIG. 11, for example, device makers' names (“Maker” in FIG. 11), device types, and workflow statuses are set as selection items. For options of the device makers' name, for example, “All,” “Device maker a,” “Device maker b,” “Device maker c,” “Device maker d,” and “Device maker e” are set. For options of the device type, for example, “All,” “IPG,” “CRT-P,” “CRT-D,” “ICD,” and “Other” are set. For options of the workflow status, for example, “All,” “Do not display “Processed”,” and “Display only unprocessed” are set. The items set as the selection items are not limited thereto. The items set as the selection items can be set from, for example, information included in the patient information stored in the memory 112 when necessary. In addition, the options for each selection item can be set in a discretional manner. In the data retrieval screen shown in FIG. 11, for example, a device maker name: “Device maker a,” device type: “CRT-P,” and workflow status: “All” are selected by a doctor or medical staff member.

Session data extracted based on the selection items is displayed in a second display region 112 on the data retrieval screen. Pieces of the session data displayed in the second display area 112 are arrayed and displayed, for example, in the order from the newest session date and time on a session-by-session basis. The session data includes the items, for example, patient ID, patient name, gender, age, device, serial number, status, description, session date and time, previous session date and time(“Prev” in FIG. 11), medical record description(“Karte dsc.” in FIG. 11), presence or absence of abnormality(“Abnormality” in FIG. 11), contact patient(“Contact” in FIG. 11), check, and save.

The display of the second display area 112 is realized through, for example, the display control function 1113 of the processing circuitry 111. For example, the processing circuitry 111 refers to the patient information stored in the memory 112 and the workflow status of XML data and excludes session data that does not correspond to the options selected in the selection items of the first display area 111 from the display target.

For the session data extracted as a display target, the processing circuitry 111 prepares each piece of display information of a patient ID, a patient name, a gender, an age, a device, a serial number, and a previous session date, based on the patient ID, patient name, gender, birth date, device name, serial number, and the last data acquisition date, included in the patient information. In addition, for the session data extracted as the display target, the processing circuitry 111 prepares each piece of display information of a status, a description, a session date and time, a medical record description, presence or absence of abnormality, contact patient, checking, save, TEXT, and PDF, based on the alert status, alert description, session date and time, description management information, abnormality checking status, contact checking status, workflow status, save management information, and link information, included in the XML data.

A device maker name: “Device maker a,” device type: “CRT-P,” and workflow status: “All” are selected on the data retrieval screen shown in FIG. 11, and the extracted session data is displayed thereon. A doctor or medical staff member checks, on the data retrieval screen shown in FIG. 11, for example, the status and/or description and enters instructions from boxes 1121 to 1124 assigned to each of the items of the medical record description, presence or absence of abnormality, contact patient, check, and save, and the save button 1125.

For example, with respect to the session data of “Patient B,” session date & time: “Aug. 31, 2018, 18:55,” the medical staff verifies the status: “Status OK,” and description: “Not applicable (N/A)”. When the medical staff determines from the contents of the status and description that there is no significant change, and it is unnecessary to contact the patient, the medical staff changes the workflow status that has been set to “Unprocessed(“Unp.” in FIG. 11)” to “Processed” in the box 1124. When the box 1124 is changed to “Processed,” a checking dialog shown in FIG. 8 is displayed on the display 114, for example. When the medical staff selects “OK”, the abnormal checking status which is set to “Not set” in the box 1122 is changed to “No significant change” (“OK” in FIG. 11), and the contact checking status which is set to “Not set” in the box 1123 is changed to “Contact unnecessary” (“Con. Unnes.” in FIG. 11).

With respect to the session data, if it is determined that presence or absence of abnormality: “No significant change” (“OK” in FIG. 11), and contact patient: “Contact unnecessary,” the medical staff determines that it is unnecessary to describe information relating to the session data in a medical record, for example. At that time, the medical staff does not tick a box 1121 indicating description management information, for example, does not enter “X” in the box, which indicates that a description in the medical record has been made.

It should be noted that even in the case of Presence or absence of abnormality: “No significant change” and Contact patient: “Contact unnecessary,” information relating to this session data may sometimes be described in the medical record. For example, a monitoring device of a given device maker transmits the data acquired for a patient intended for remote monitoring to a data center once a day. That is, 30 pieces of data are transmitted in one month. In the case of a patient who has a normal benign course, it may result in the presence or absence of abnormality for 30 days in succession: “No significant change” and contact patient: “Contact unnecessary”. Even in such a case, in order to record that instructions necessary for medical treatment were provided at least once a month, a box indicating description management information is ticked, with for example an “X”, which indicates that a description in a medical record has been entered.

In addition, with respect to the session data, when it is determined that Presence or absence of abnormality: “No significant change” and Contact patient: “Contact unnecessary”, the medical staff determines that it is unnecessary to output PDF data and text data on this session data to the department server 12 and the hospital information system 13, for example. At that time, the medical staff does not press down the save button 1125 for which “Save” is set as the default.

Even in the case of Presence or absence of abnormality: “No significant change” and Contact patient: “Contact unnecessary”, data on this session data may sometimes be output. For example, even in the case of Presence or absence of abnormality: “No significant change” and Contact patient: “Contact unnecessary,” a save button for which “Save” is set as the default is pressed in accordance with at least one time of guidance a month.

On the data retrieval screen shown in FIG. 11, for example, with respect to session data in which Patient name: “Patient E” and Session date & time: “Aug. 31, 2018; 16:05”, Check: “Dr. is checking” (“Dr.Chkin'” in FIG. 11) is set. At that time, the doctor is judging the presence or absence of abnormality, whether or not contacting the patient is required, whether or not a description in a medical record is to be done, and whether or not PDF data or text data for this session data is to be downloaded, from another receiving apparatus 11, for example. For example, when the doctor would also like to check past session data on the patient E, the doctor selects Patient name: “Patient E” shown in FIG. 11. When Patient name: “Patient E” is selected, an individual screen for the patient E is displayed at the receiving apparatus 11 which is being used by the doctor.

FIG. 12 is a pattern diagram depicting an example of an individual screen on a patient-by-patient basis transitioned from the data retrieval screen shown in FIG. 11. The individual screen shown in FIG. 12 depicts an individual screen in the case where the patient E is selected in FIG. 11, for example. The receiving apparatus 11 can prepare a similar individual screen for a discretional patient selected by a doctor or medical staff member.

On the individual screen shown in FIG. 12, comments on the patient E entered by a doctor or medical staff member are displayed on a first display area 121. The comments displayed in the first display area 121 are displayed through, for example, the display control function 1113, based on the comment information in the patient information stored in the memory 112. Writing comments in the first display area 121 is performed by the doctor or medical staff member by pressing a comment insertion button 122. That is, through pressing the comment insertion button 122, the comment management function 1115 is executed, and for example, a dialog box for insertion of comments pops up. The doctor or medical staff member writes notes, etc. as comments in the popped-up dialog box by pressing the comment insertion button 122.

It should be noted that the insertion of comments is not limited to entries via a dialog box. For example, the individual screen may be configured such that a comments insertable area is provided at the lower end of the first display area, and when comments are entered in this area, the entered comments are reflected in the first display area 121. In addition, comments entered after logging in by one user and comments entered by another person may be displayed in a distinguishable manner. For example, comments logged in and entered by one user may be displayed close to the right side of the first display area 121, and comments entered by another person may be displayed close to the left side. Furthermore, comments logged in and entered by one user and comments entered by another person may be displayed so as to be distinguished by using different colors.

Written comments are stored in the patient information stored in the memory 112. By displaying comments on the display screen in this way, it becomes possible to efficiently share individual information of a patient as well as notes, etc. of the patient. In a case where processing is carried over to the next day, items to be reported or items to be suspended are efficiently performed. In addition, it becomes also possible to efficiently confirm items to be reported, etc.

Session data for a designated patient acquired in the past is displayed in a second display area 123 on the individual screen. The pieces of session data displayed in the second display area 123 are arrayed and displayed, for example, in the order from the newest session date and time on a session-by-session basis. The session data includes items of, for example, session date and time, status, description, measurement/statistic summary, medical record description, presence or absence of abnormality, contact patient, check, save, TEXT, and PDF.

The display of the second display area 123 is realized through the display control function 1113 of the processing circuitry 111. With respect to session data of a selected patient included in XML data, the processing circuitry prepares, for example, each piece of display information of session date and time, status, description, medical record description, presence or absence of abnormality, contact patient, check, save, TEXT, and PDF, based on the session date and time, alert status, alert description, description management information, abnormality checking status, contact checking status, workflow status, and save management information. Furthermore, with respect to the session data of the selected patient, the processing circuitry 111 prepares display information of measurement/statistic summary based on a plurality of parameters included in data tables, such as AF/AT, pacing rate, and lead sensing information.

In the second display area 123, a box 1231 provided in the measurement/statistic summary indicates kinds of parameters displayed in the measurement/statistic data. When pressing the box 1231, a doctor or medical staff member selects, for example, a desired kind of parameter from the kinds of parameters pull-down menu displayed. The processing circuitry 111 extracts a parameter of the kind selected from a data table, for example, and prepares display information of the measurement/statistic summary based on the extracted parameter.

In the second display area 123 shown in FIG. 12, AT/AF is selected. The processing circuitry 111 extracts the parameter of the AT/AF selected from the data table and prepares the display information of measurement/statistic summary for each piece of session data, based on the extracted AT/AF parameter.

The kind of parameter selected by a doctor or medical staff member is stored in the patient information as contents of the last displayed measurement/statistic data. The contents of the last displayed measurement/statistic data stored in the patient information are read out when the screen is next transitioned to the individual screen. The contents of the last displayed measurement/statistic data are displayed on the next transitioned individual screen without waiting for a selection of the doctor or medical staff member. This allows the doctor or medical staff member to focus attention on a particular item and easily ascertain the trend situation of the patient.

The doctor or medical staff member refers to past session data shown in the second display area 123 of FIG. 11 to determine the presence or absence of abnormality, whether or not contacting the patient is required, whether or not a description in the medical record is required, and whether or not PDF data or text data is to be downloaded. The doctor or medical staff member enters instructions in boxes for the medical treatment description, presence or absence of abnormality, patient checking, and checking, and a save button for data save, based on the determination result. The processing circuitry 111 further updates the XML data based on the entries from the doctor or medical staff member and changes the displays of the boxes for the medical treatment description, presence or absence of abnormality, patient checking, and check, and the save button for data-save.

For example, it is assumed that with respect to the latest session data shown in FIG. 12, the doctor or medical staff member determines that it is necessary to describe information on the session data in a medical record. At that time, the doctor or medical staff member ticks the box 1232 or enters “X” meaning that he or she will perform (or performed) a description in the medical record in the box 1232 indicating the description management information. When “X” is entered in the box 1232, the processing circuitry 111 changes the description management information in the XML data to “Perform”.

When “X” is entered in the box 1232, a copy button appears, for example, on the lower side of the box 1232, and the doctor or medical staff member can press down it. For example, when the copy button is pressed, the processing circuitry 111 executes the duplication function 1114. In the duplication function 1114, the processing circuitry 111 prepares text information according to the set abnormal checking status. For example, the processing circuitry 111 prepares text information in which the date and time at which the box 1232 was ticked and the name of person (login name) are added to a fixed phrase preliminarily set according to the abnormal checking status.

Specifically, in the case where the abnormality checking status is “No significant change”, the processing circuitry 111 prepares text information in which the date and time at which the box 1232 was ticked and the name of person, for example, “2018/08/31 16:10 “Person in charge: AA AA” are added to a given fixed phrase: “Performed evaluations including measurements of functional indexes of cardiac implantable device by remote monitoring, and no significant change was found.” When the abnormality checking status indicates “Checked”, the processing circuitry 111 prepares text information in which the date and time at which the box 1232 was ticked and the name of person, for example, “2018/08/31 16:10 “Person in charge: AA AA” are added to a given fixed phrase: “Performed evaluations including measurements of functional indexes of cardiac implantable device by remote monitoring, and the following matter was checked.” The date and time at which the box 1232 was ticked and the name of the person who performed it may be the date and time and the name of person when the copy button is pressed by a doctor or medical staff member.

For example, when text data for the latest session data shown in FIG. 12 is output to the department server 12 and/or hospital information system 13, the doctor or medical staff member presses a TXT button 1233, for example. When the TXT button 1233 is pressed, the processing circuitry 111 reads out link information of text data included in the XML data and downloads the text data from the cloud system 20 based on the read out link information. The processing circuitry 111 outputs the downloaded text data in a file format or CSV format to the department server 12 and/or hospital information system 13.

For example, in the case where PDF data for the latest session data shown in FIG. 12 is output to the department server 12 and/or hospital information system 13, the doctor or medical staff member presses a PDF button 1234, for example. When the PDF button 1234 is pressed, the processing circuitry 111 reads out link information of PDF data included in the XML data and downloads the PDF data from the cloud system 20 based on the read out link information. The processing circuitry 111 starts up, for example, a PDF reader to display the downloaded PDF data.

When the text data and/or PDF data is downloaded, the doctor or medical staff member presses a save button 1235 and enters instructions to the effect that the downloaded data should be saved. When the save button 1235 is pressed, the processing circuitry 111 outputs the downloaded text data and/or PDF data to a folder preliminarily designated based on, for example, a configuration, etc., through the data output function 1116. When the data is output to the given folder, the processing circuitry 111 changes the save management information in the XML data to “Saved” and changes the display of the save button 1235 to “Saved”. The department server 12 and/or hospital information system 13 monitor an output of text data and/or PDF data to a given folder, for example. When the department server 12 and/or hospital information system 13 detects an output of text data and/or PDF data to a given folder, it accesses the folder and reads out the text data and/or PDF data stored in the folder. The processing circuitry 111 may output the downloaded text data and/or PDF data to, for example, the department server 2 and/or hospital information system 13 through the data output function 1116.

When an operation history button 124 is pressed on the individual screen shown in FIG. 12, an area for displaying an operation log (not shown) is displayed. The display information on the operation log displayed at that time is prepared based on the operation history of patient information, for example. By enabling browsing of the operation history, all of the operations linked to the patient can be checked.

FIG. 13 is a pattern diagram showing an example of a screen for doctors transitioned from the home screen shown in FIG. 10. Among the session data stored in the memory 112, the session data that has passed checking by medical staff and becomes a status of asking a doctor to check details is displayed on the screen for doctor.

Selection items for selecting an extraction target are displayed in a first display area 131 on the screen for doctor. Specifically, for example, items based on a workflow status are set in the first display area 131 shown in FIG. 13. As options of the items based on the workflow status, for example “Display only “Request Dr.” (New: 2; Urgent: 1)” and “Display request Dr.” and “Dr. is processing” are set. It should be noted that any options can be set for the selection items. On the screen for doctors shown in FIG. 13, for example, the workflow status: “Display “Request Dr.” and “Dr. is processing”” are selected by the doctor.

The session data extracted based on the selection items is displayed in a second display area 132 on the screen for doctors. The pieces of session data displayed in the second display area 132 are arrayed and displayed, for example, in the order from the newest session date and time on a session-by-session basis. The session data includes a patient ID, patient name, gender, age, device, serial number, status, description, session date and time, the previous session date, check, comments, TEXT, and PDF, for example.

The display of the second display area 132 is realized through, for example, the display control function 1113 of the processing circuitry 111. For example, the processing circuitry 111 refers to the workflow status of the XML data and excludes session data which is not applicable to the options selected in the selection items of the first display area 131 from display targets.

For the session data extracted as a display target, the processing circuitry 111 prepares each piece of display information of a patient ID, a patient name, a gender, an age, a device, a serial number, and a previous session date, based on the patient ID, patient name, gender, birth date, device name, serial number, and the last data acquisition date, included in the patient information. Also, for the session data extracted as a display target, the processing circuitry 111 prepares display information on each of the status, description, session date and time, checking, TEXT and PDF, based on the alert status, alert description, session date and time, workflow status, and link information, included in the XML data. The comment display button is a button for extending the display area of comments written for a patient. When a cursor overlaps at the comment display button, the processing circuitry 111 may pop up the latest comments for only the number of preliminarily set cases.

The workflow status: “Display “Request Dr.” (“Req.Dr.” in FIG. 13) and “Dr. is processing” (“Dr.Chkin'” in FIG. 13) is selected and the extracted session data is displayed on the screen for doctors shown in FIG. 13. At that time, the session data for which the workflow status: “Urgent” (displayed as “Request Dr. (Urgent)” in FIG. 5) is set is prioritized over the other session data and displayed on the first line in a highlighted manner.

The doctor checks the session data for which the workflow status: “Urgent,” “Request Dr.” and “Dr. is processing” are set, for example, on the screen for doctors shown in FIG. 13. The doctor selects, for example, Patient: “Patient B” of the session data for which the workflow status: “Urgently request” is set, and transitions the screen for doctors to the individual screen for the patient B. The doctor refers to the past session data on the individual screen for the patient B to determine the presence or absence of abnormality, whether or not contacting the patient is required, whether or not a description in the medical record is required, and whether or not PDF data or text data is to be downloaded. The items of a medical record description, presence or absence of abnormality, contact patient, check, save, TEXT, and PDF may be displayed on the screen for doctors to let a doctor determine, on the screen for doctors, the presence or absence of abnormality, whether or not contacting the patient is required, whether or not a description in the medical record is required, and whether or not PDF data or text data is to be downloaded.

FIG. 14 is a pattern diagram showing an example of a data checking screen transitioned from the home screen shown in FIG. 10. At least part of the session data stored in the memory 112 is displayed on the data checking screen.

The selection items for selecting an extraction target are displayed in a first display area 141 on the data checking screen. Specifically, in the first display area 141 shown in FIG. 14, for example, items based on the alert status and workflow status are set. For example, “Display only newly arriving alert (Red: 4; Yellow: 3)”, and “display “Newly arriving alert “unprocessed”+“M staff is processing” and “Request M staff”” are set for options of the selection items. It should be noted that any options can be set for the selection items. For example, “Display “Newly arriving alert “unprocessed”+“M staff is processing” and “Request M staff”” are selected on the data checking screen shown in FIG. 14.

The pieces of session data extracted based on the selection items are displayed in a second display area 142 on the data checking screen. The pieces of session data displayed in the second display area 142 are arrayed and displayed in the order of the newest session date and time on a session-by-session basis. The session data includes a patient ID, patient name, gender, age, device, serial number, status, description, session date and time, the previous session date, check, comments, TEXT, and PDF, for example.

The display of the second display area 142 is realized through the display control function 1113 of the processing circuitry 111, for example. For example, the processing circuitry 111 refers to the alert status and workflow status of the XML data and excludes session data which is not applicable to the options selected in the selection items of the first display area 141 from display targets.

For the session data extracted as a display target, the processing circuitry 111 prepares each piece of display information of a patient ID, a patient name, a gender, an age, a device, a serial number, and a previous session date, based on the patient ID, patient name, gender, birth date, device name, serial number, and the last data acquisition date, included in the patient information. Also, for the session data extracted as the display target, the processing circuitry 111 prepares display information on each of the status, description, session date and time, check, TEXT and PDF, based on the alert status, alert description, session date and time, workflow status, and link information, included in the XML data.

“Display “Newly arriving alert “Unprocessed”+“M staff is processing” and “Request M staff”” is selected on the data checking screen shown in FIG. 14, and the extracted session data is displayed thereon. A medical staff member promptly checks, for example, the session data presented on the data checking screen shown in FIG. 14 and determines whether or not the session data is applicable to “Request doctor,” “Request doctor (Urgent),” or “Processed”. This allows medical staff to check, for example, the session data for which an alert is issued, promptly and on a priority basis. This also allows the medical staff to efficiently access the session data for which “Medical staff (M staff) is processing” is set even in the case where the medical staff stops the task once along the way and then returns to the task.

When determining the presence or absence of an abnormality, whether or not contacting the patient is required, whether or not a description in the medical record is required, and whether or not PDF data or text data is to be downloaded, the medical staff member selects, for example, a patient: “Patient C” possessing the session data for which the workflow status: “Unprocessed” is set and transitions the data checking screen to the individual screen for the patient C. The medical staff refers to the past session data on the individual screen for the patient C to determine the presence or absence of abnormality, whether or not contacting the patient is required, whether or not a description in the medical treatment record is required, and whether or not PDF data or text data is to be downloaded.

FIG. 15 is a pattern diagram showing an example of a no-data patient checking screen transitioned from the home screen shown in FIG. 10. Among the patients stored in the memory 112, patients for whom there is received data within a given period of time but there is no received data over a specified period are displayed on the no-data patient checking screen.

Selection requirements for selecting extraction targets and selection items are displayed in a first display area 151 on the no-data patient checking screen. Specifically, in the first display area 151 shown in FIG. 15, for example, “Patient for whom there is received data within 13 months in the past but there is no received data within 3 weeks” is set as a selection condition. It should be noted that the period of three weeks is selectable from a pull-down menu. The selected period is succeeded on the next display. In addition, in the first display area 151 shown in FIG. 15, for example, the presence or absence of display for a patient for whom follow-up was stopped is set as a selection item. The no-data patient checking screen shown in FIG. 15 is set such that for example, a patient for whom follow-up was stopped by a doctor or medical staff member is not displayed on the screen.

Pieces of patient data extracted based on the selection requirements and selection items are displayed in a second display area 152 on the no-data patient checking screen. The pieces of patient data displayed in the second display area 152 are arrayed and displayed in the order from the longest elapsed time on a patient-by-patient basis, for example. The patient data includes items of a patient ID, patient name, gender, age, device, serial number, elapsed time, the previous session date, follow-up setting, check, comments, TEXT, and PDF, for example.

The display of the second display area 152 is realized through, for example, the display control function 1113 of the processing circuitry 111. For example, the processing circuitry 111 refers to the patient information stored in the memory 112 and the XML data and excludes patient data that is not applicable to the option selected in the selection items of the first display area 151.

With respect to the patient data extracted as a display target, the processing circuitry 111 prepares each of the display information on a patient ID, patient name, gender, age, device, serial number, elapsed time, the previous session date, and follow-up setting, based on the patient ID, patient name, gender, birth date, device name, serial number, the last data acquisition date, and presence or absence of follow-up, included in the patient data. In the follow-up setting, for the patient whom follow-up has been continued, a button 1521 for entering instructions to stop follow-up is provided.

Patient data for patients for whom follow-up is still continued is displayed on the no-data patient checking screen shown in FIG. 15. In the case where there is a patient for whom follow-up is stopped for the reason, for example, that the patient passed away or changed hospital, etc., a doctor or medical staff member presses the button 1521 on the no-data patient checking screen shown in FIG. 15 to stop follow-up. When a button 1521 is pressed, the processing circuitry 111 changes the presence or absence of follow-up in the patient information to “Stop follow-up”.

In the case where, for example, the no-data patient checking screen shown in FIG. 15 is set to also display patients for whom follow-up is stopped, for example, a button for entering instructions to the effect that follow-up will be resumed is provided in the follow-up setting for the patients of “Stop follow-up”. A doctor or medical staff member presses the button for a patient for whom follow-up should be resumed. When the button to the effect that follow-up will be resumed is pressed, the processing circuitry 111 changes the presence or absence of follow-up in the patient information to “With follow-up”.

By providing a requirement of “There is received data within past N months”, a list of devices for the patient to receive a physical examination and a ledger of patients are not required, allowing simplification of the system.

FIG. 16 is a pattern diagram showing an example of a description status checking screen transitioned from the home screen shown in FIG. 10. A description status in a medical record is displayed for each patient on a monthly basis on the description status checking screen.

Selection items for selecting an extraction target are displayed in the first display area 161 on the description status checking screen. Specifically, for example, items based on the description status in a medical record are set as selection items in the first display area 161 shown in FIG. 16. For options of the selection items, for example, “All,” “Display patient indicated by “x”,” “Display patient indicated by “ok”,” and “Display patient indicated by “F”” are set. It should be noted that “x” indicates a month during which no description is present, “ok” indicates a month determined by a doctor or medical staff member that monitoring may not be performed, and “F” indicates a month in which the patient visited the hospital for the purpose of follow-up. It should be noted that any options can be set for the selection items. On the description status checking screen shown in FIG. 16, for example, “All” is selected by a doctor or medical staff member.

Pieces of patient data extracted based on the selection items are displayed in a second display area 162 on the description status checking screen. The pieces of patient data displayed in the second display area 162 are arrayed and displayed on a patient basis in a given order. Patient data includes the items, for example, patient ID, patient name, device, serial number, and monthly description status of one-year.

The display of the second display area 162 is realized through the display control function 1113 of the processing circuitry 111. For example, the processing circuitry 111 refers to description management information of the XML data and excludes patient data that is not applicable to the option selected in the selection items of the first display area 161 from display targets.

With respect to the patient data extracted as the display targets, the processing circuitry 111 prepares display information on each of a patient ID, patient name, device, and serial number, based on the patient ID, patient name, device name, and serial number, included in the patient information. Also, with respect to the patient data extracted as the display target, the processing circuitry 111 prepares display information on a monthly description status for one year, based on the number of portions where “Perform” is set in the description management information included in the XML data.

On the description status checking screen shown in FIG. 16, pieces of data for all patients indicated by “x,” “ok,” and “F” are displayed. A doctor or medical staff member refers to the number of displayed times on a monthly basis, “x,” “ok,” and “F” and checks whether or not missing information is present in the medical record. When the display “x” indicating that no description is present in the medical record is modified, the doctor or medical staff member presses “x”, for example. When “x” is pressed, a dialog 1621 is displayed, for example. Changes in status to “x,” “ok,” and “F” are displayed in the dialog 1621. A doctor or medical staff member selects one from “x,” “ok,” and “F” and presses OK to fix the change.

As described above, in the present embodiment, the receiving apparatus 11 is configured to set, in session data acquired from an implantable device, a workflow status which can be branched along the transitions of a person in charge of checking data in accordance with the status of data. With this configuration, it is possible to efficiently transition a roll of monitoring processing of session data between doctors and medical staff members.

Also, in the present embodiment, the receiving apparatus is configured to set an abnormality checking status indicating a checking route of abnormality for the session data acquired from an implantable device. With this configuration, it becomes possible to efficiently manage within a team whether or not abnormality occurs in the monitored session data.

Also, in the present embodiment, the receiving apparatus 11 is configured to set, in session data acquired from an implantable device, a contact checking status indicating a checking route of whether or not contacting the patient is required. With this configuration, it is possible to efficiently manage within a team whether or not the contents of session data should be reported to the patient and whether information such as a request for visiting the hospital has been reported to the patient.

Also, in the present embodiment, the receiving apparatus 11 is configured to include an alert status in session data acquired from an implantable device. With this configuration, it becomes possible to efficiently manage alert histories.

Also, in the present embodiment, the receiving apparatus 11 is configured to set, in session data acquired from an implantable device, description management information for managing the presence or absence of a description in a medical record. With this configuration, it becomes possible to efficiently manage descriptions in a medical record.

In addition, in the present embodiment, the receiving apparatus 11 is configured to set, in session data acquired from an implantable device, save management information for managing whether or not text data and/or PDF data has been downloaded. With this configuration, it becomes possible to readily manage the presence or absence of downloading.

Also, in the present embodiment, the receiving apparatus 11 is configured to display matters to be addressed on a priority basis, based on the workflow status on the display 114. With this configuration, the items to be checked can be checked at a glance.

Also, in the present embodiment, the receiving apparatus 11 is configured to display, on the display 114, a screen corresponding to a matter selected in accordance with the selection for the matter to be addressed on a priority basis. That is, it becomes possible to directly transition from the home screen to the screen for doctors, for example. This allows the receiving apparatus 11 to narrow down data for a patient who is necessary to be referenced or addressed and to provide the data to a doctor, for example.

Furthermore, it becomes possible to directly transition from the home screen to the data checking screen, for example. This allows the receiving apparatus 11 to narrow down data for a patient who needs to be referenced or addressed, for example, to provide the data to medical staff. In addition, since the efficiency of reference increases, it become possible for the medical staff to request a doctor to promptly provide treatment for the patient.

In addition, it becomes possible to directly transition from the home screen to the no data patient checking screen, for example. This allows the receiving apparatus 11 to narrow down data for patients whose remote data should have been imported but has not been taken in so as to provide the data to a doctor or medical staff member. A patient's adherence to remote monitoring is very important for ascertaining the status of the patient, intervention of treatment, reduction in death rate, and maintaining QOL. The patient's adherence is also an important element for the revenue of the hospital. Allowing the narrowing-down of data of a patient whose remote data should have been taken in but was not taken in and providing the data makes it possible for doctors and medical staff members to respect the patient's adherence.

In addition, in the present embodiment, the receiving apparatus 11 is configured to display, on the display 114, a description status in a medical record on a patient-by-patient basis, based on the description management information. This allows doctors and medical staff members to efficiently check the description status to the medical record.

It should be noted that in the above-described embodiment, an example is described in which medical staff includes clinical engineers and nurses. However, nurses may be treated as staff that is independent of medical staff. In recent years, the number of cases of implementing remote monitoring has increased, and the scale of implementation of remote monitoring in medical facilities is expanding. With the expansion in scale of the implementation of remote monitoring, telephonic contact for a patient and a diagnostic interview via a telephonic contact, and ascertainment of the health condition (interrogation), etc. have also become important. In addition, interrogation prior to a visit to a doctor in the follow-up of a patient who has visited a hospital has also become important. Therefore, there exist facilities where a nurse who directly responds to a patient in a remote monitoring task is referred to as a “device nurse”, and the device nurse is assigned as an elected and appointed nurse.

FIG. 17 is a pattern diagram showing a setting example of a workflow status in the case where a nurse is treated as staff that is independent of medical staff. In the description of FIG. 17, medical staff includes clinical engineers, for example. For example, when acquiring data from the cloud system 20, the processing circuitry 111 executes the data setting function 1112 and sets the workflow status to “Unprocessed” as the default.

The workflow status of “Unprocessed” transitions to “M staff is processing” in accordance with instructions entered by medical staff, for example. Also, the workflow status of “Unprocessed” transitions to “Nurse is processing” in accordance with instructions entered by a nurse, for example.

The workflow status of “M staff is processing” transitions to “Processed,” “Request Dr. (Urgent)” or “Request Dr.” in accordance with instructions entered by medical staff, for example. The workflow status of “Nurse is processing” transitions to “Processed,” “Request Dr. (Urgent),” or “Request Dr.” in accordance with instructions entered by the nurse, for example.

The workflow statuses of “Request Dr. (Urgent)” and “Request Dr.” transition to “Dr. is processing” in accordance with instructions entered by a doctor, for example. The workflow status of “Dr. is processing” transitions to “Processed,” “Request M staff,” or “Request nurse” in accordance with instructions entered by the doctor, for example.

The workflow status of “Request M staff” transitions to “M staff is processing” in accordance with instructions entered by medical staff, for example. The workflow status of “Request nurse” transitions to “Nurse is processing” in accordance with instructions entered by a nurse, for example. It should be noted that the transition of a workflow status is not limited to the transition shown in FIG. 17. For example, a workflow status may transition from “M staff is processing” to “Request nurse” or may transition from “Nurse is processing” to “Request M staff”.

According to at least one embodiment of the embodiments described above, the receiving apparatus 11 can reduce loads of doctors and hospital staff in instructing a patient using remote monitoring.

The wording of “processor” used in the descriptions of the embodiments means, for example, a central processing unit (CPU), a graphics processing unit (GPU), or a circuit such as an application-specific integrated circuit (ASIC), a programmable logic device (e.g., simple programmable logic device (SPLD), and a complex programmable logic device (CPLD), and a field programmable gate array (FPGA)). The processor reads out a program stored in a storage circuit and executes it, thereby realizing a function. Instead of storing a program in the storage circuit, it may be configured such that the program is directly incorporated into the circuit of the processor. In this case, the processor realizes the function by reading the program incorporated into the circuit and executing it. It should be noted that each of the processors in the respective embodiments described above is not limited to the case of being configured as a single circuitry for each processor, and a plurality of independent processors may be combined together as one processor to realize the function. Furthermore, a plurality of structural components in the respective embodiments described above may be integrated into one processor to realize the function.

Moreover, the functions described in connection with the above embodiment may be implemented, for example, by installing a program for executing the processing in a computer, such as a workstation, etc., and expanding the program in a memory. The program that causes the computer to execute the processing can be stored and distributed by means of a storage medium, such as a magnetic disk (a hard disk, etc.), an optical disk (CD-ROM, DVD, etc.), and a semiconductor memory.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An information management system comprising:
an acquisition apparatus which acquires data stored in an external storage;
a commonalization apparatus which converts the acquired data into a preliminarily set commonalization format; and
a receiving apparatus which receives the data converted into the commonalization format and sets, in the received data, a workflow status capable of branching along a transition of a person in charge of checking data in accordance with a status of the data.

2. The information management system according to claim 1, wherein the receiving apparatus sets, in the received data, an abnormality checking status indicating an abnormality checking route.

3. The information management system according to claim 1, wherein the receiving apparatus sets, in the received data, a contact checking status indicating a checking route of whether or not contacting a patient is required.

4. The information management system according to claim 1, wherein the receiving apparatus sets, in the received data, description management information for managing the presence or absence of a description in a medical record.

5. The information management system according to claim 1, wherein the receiving apparatus sets, in the received data, save management information for managing whether or not data associated with the received data is saved.

6. The information management system according to claim 1, wherein the receiving apparatus displays, on a display, a matter to be addressed on a priority basis, based on the workflow status.

7. The information management system according to claim 6, wherein the receiving apparatus displays, on the display, a screen corresponding to the matter to be addressed on a priority basis in accordance with a selection of the matter.

8. The information management system according to claim 5, further comprising:
a text preparation apparatus which prepares text data based on the data converted into the commonalization format,
wherein when the receiving apparatus is housed in an intra-hospital system and outputs the text data prepared by the text preparation apparatus to a device of the intra-hospital system, the receiving apparatus inputs in the save management information that the data associated with the received data is saved.

9. The information management system according to claim 5, wherein the acquisition apparatus acquires detailed information from the external storage, and when the receiving apparatus is housed in an intra-hospital system and outputs the detailed information acquired by the acquisition apparatus to a device of the intra-hospital system, the receiving apparatus inputs in the save management information that the data associated with the received data is saved.

10. The information management system according to claim 4, wherein the receiving apparatus displays, on a display, a status of a description in the medical record on a patient-by-patient basis, based on the description management information.

11. A receiving apparatus comprising
processing circuitry configured to:
acquire data stored in a storage whose security is assured and
set, in the acquired data, a workflow status capable of branching along transitions of a person in charge of checking data in accordance with a status of the data.

12. The receiving apparatus according to claim 11, wherein the processing circuitry sets, in the acquired data, an abnormality checking status indicating an abnormality checking route.

13. The receiving apparatus according to claim 11, wherein the processing circuitry sets, in the acquired data, a contact checking status indicating a checking route of whether or not contacting a patient is required.

14. The receiving apparatus according to claim 11, wherein the processing circuitry sets, in the acquired data, description management information for managing the presence or absence of a description in a medical record.

15. The receiving apparatus according to claim 11, wherein the processing circuitry sets, in the acquired data, save management information for managing whether or not data associated with the acquired data is saved.

16. The receiving apparatus according to claim 11, wherein the processing circuitry displays, on a display, a matter to be addressed on a priority basis, based on the workflow status.

17. The receiving apparatus according to claim 16, wherein the processing circuitry displays, on the display, a screen corresponding to the matter to be addressed on a priority basis in accordance with a selection of the matter.

18. The receiving apparatus according to claim 15, wherein the processing circuitry acquires text data prepared based on the acquired data and stored in the storage,
when the processing circuitry outputs the text data to a device of an intra-hospital system, the processing circuitry inputs in the save management information that data associated with the received data is saved.

19. The receiving apparatus according to claim 15, wherein the processing circuitry acquires detailed information stored in the storage, when the processing circuitry outputs the detailed information to a device of an intra-hospital system, the processing circuitry inputs in the save management information that data associated with the received data is saved.

20. The receiving apparatus according to claim 14, wherein the processing circuitry displays, on a display, a state of a description in the medical record on a patient-by-patient basis, based on the description management information.
